(19) United States
(12) Patent Application Publication
(10) Pub. No.: US 2026/0354959 A1
(43) Pub. Date: Nov. 12, 2026

(54) Protective Mesh for Tissue Constructs

(71) Applicant: President and Fellows of Harvard College, Cambridge, MA (US)

(72) Inventors: Sebastien Guy Marcel UZEL, Cambridge, MA (US); Luba PERRY, Cambridge, MA (US); Jonathan Eric RUBINS, Cambridge, MA (US); Jennifer A. LEWIS, Cambridge, MA (US)

(21) Appl. No.: 19/475,286

(22) PCT Filed: Apr. 18, 2024
(86) PCT No.: PCT/US2024/025107

§ 371 Date: Oct. 14, 2025

Related U.S. Application Data
(60) Provisional application No. 63/497,070, filed on Apr. 19, 2023.
(60) Provisional application No. 63/500,450, filed on May 5, 2023.

(51) Int. Cl. A61F 2/00

(52) U.S. Cl. CPC A61F 2/0063; A61F 2002/0081; A61F 2210/0004; A61F 2220/0008; A61F 2220/0091

(57) ABSTRACT

A structural mesh for a tissue construct may comprise a mesh casing shaped to partially or fully enclose a tissue construct. The mesh casing may comprise a mesh material having a stiffness sufficient for the mesh casing to provide structural support while the tissue construct sustains internal pressure from blood circulation. Pores in the mesh casing may allow for transport of oxygen and nutrients between the tissue construct and a surrounding environment.

### RELATED APPLICATIONS

The present patent document claims the benefit of priority to U.S. Provisional Patent Application No 63/500,450, filed on May 5, 2023, and to U.S. Provisional Patent Application No. 63/497,070, filed on Apr. 19, 2023. Both of the aforementioned patent applications are hereby incorporated by reference in their entirety.

### TECHNICAL FIELD

This disclosure relates generally to biomedical devices and more particularly to a biomedical device for supporting a tissue construct when being anastomosed to a host vasculature in vivo or when being perfused in vitro.

### BACKGROUND

Despite advances in research and increased awareness of organ donation and transplantation, there continues to be a gap between the need for organs and available organ donations. Currently, more than 100,000 men, women and children are waiting for lifesaving organ transplants. Every ten minutes in the U.S., another person is added to the national transplant waiting list, and 7,000 deaths occur every year in the U.S. because organs are not donated in time. It would be beneficial to develop alternative technologies to enable organ transplantation to take place without waiting for donor organs.

The emerging ability to engineer three-dimensional (3D) vascularized tissues on demand may enable scientific and technological advances in tissue engineering, drug screening, toxicology, 3D tissue culture, organ repair, and organ transplantation. To produce 3D tissue constructs that mimic natural tissues and, ultimately, organs, several key components—cells, extracellular matrix (ECM), and vasculature—may be assembled in complex arrangements. Each of these components plays a vital role: cells are the basic unit of all living systems, ECM provides structural support, and vascular networks provide efficient nutrient and waste transport, temperature regulation, delivery of factors, and long-range signaling routes. Without perfusable vasculature within a few hundred microns of each cell, three-dimensional tissues may quickly develop necrotic regions. Recently, progress has been made in embedding vascular networks in tissue constructs via 3D printing, as described for example in U.S. Pat. No. 10,117,969 to Lewis et al., entitled “Method of Printing a Tissue Construct with Embedded Vasculature,” which is hereby incorporated by reference. In order to successfully employ such vascularized tissue constructs, for example, in in vivo or in vitro applications, it would be advantageous to develop a mechanism or system to support the tissue construct when it is integrated into a host organ or vasculature or being perfused in vitro.

### BRIEF SUMMARY

A structural mesh for a tissue construct comprises a mesh casing shaped to partially or fully enclose a tissue construct. The mesh casing comprises a mesh material having a stiffness sufficient for the mesh casing to provide structural support while the tissue construct sustains internal pressure from blood circulation or perfusion of cell culture medium. Pores in the mesh casing allow for transport of oxygen and nutrients between the tissue construct and a surrounding environment. A structural mesh system may comprise a tissue construct. The tissue construct may include a vascular channel. The mesh casing may partially or fully enclose the tissue construct. In one application, a suturable cuff may be anchored to the tissue construct. The suturable cuff may be configured for integration with one or more body vessels, or any type of bodily duct. The structural mesh system may be disposed in the body of a patient. One or more of the body vessels may be in fluid communication with the suturable cuff or directly with tissue bodies.

A method of supporting the tissue construct in a host organ, vasculature, tissue body, or culture medium is described below. The tissue construct and structural mesh may be used, for example, in an in vivo or an in vitro application. The method may comprise placing a tissue construct which includes a vascular channel inside of a mesh casing, such that the mesh casing partly or fully surrounds the tissue construct. The mesh casing and the tissue construct may then be inserted into a body of a patient or into a culture medium and perfused with blood or another bodily fluid. During the perfusion, the mesh casing provides support to the tissue construct to withstand a pressure of the blood or other bodily fluid being perfused into the tissue construct.

In another application, the method further comprises providing the suturable cuff. The suturable cuff comprises a hollow body having an anastomotic end, an anchoring end, and one or more lumens. Each lumen extends through the hollow body from a proximal opening at the anastomotic end to a distal opening at the anchoring end. The method comprises anchoring the tissue construct including a vascular channel to the anchoring end of the suturable cuff. The vascular channel is positioned to be in fluid communication with the distal openings. The method comprises anastomosing one or more body vessels to the anastomotic end of the suturable cuff, whereby the one or more body vessels are in fluid communication with the proximal openings; perfusing the suturable cuff and the anchored tissue construct with blood or another bodily fluid from the patient.

### BRIEF DESCRIPTION OF THE DRAWINGS

The embodiments may be better understood with reference to the following drawings and description. The components in the figures are not necessarily to scale. Moreover, in the figures, like-referenced numerals designate corresponding parts throughout the different views.

FIGS. 1A and 1B illustrate a schematic of an example structural mesh system in use;

FIGS. 2A-2B illustrate example designs of a mesh casing;

FIGS. 3A-3C illustrate an example of a “clamshell” design of a mesh casing;

FIGS. 4A-4D illustrate an example of an organ-specific geometry design of a mesh casing; and

FIGS. 5A-5E illustrate an example of an application of the structural mesh system on a tissue construct.

### DETAILED DESCRIPTION

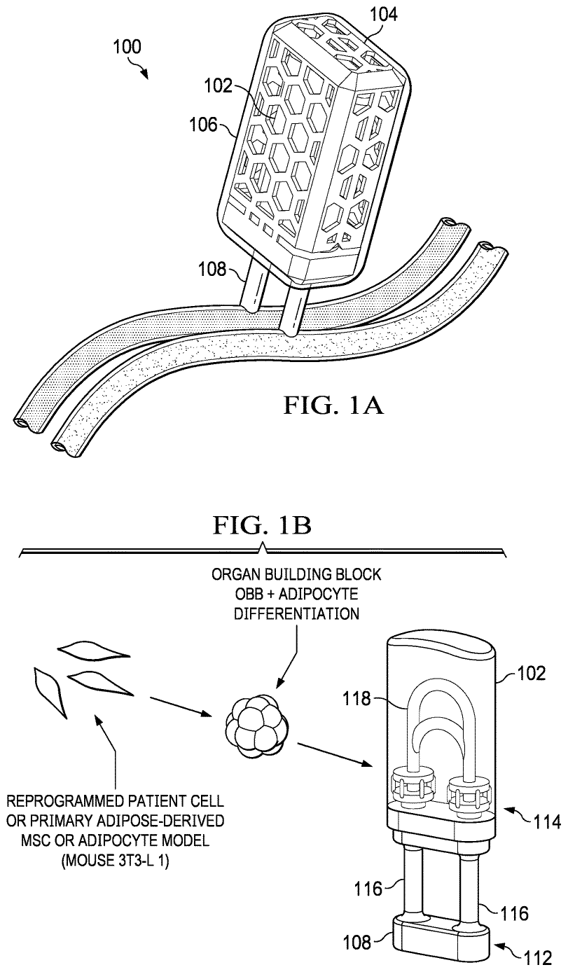

FIG. 1A illustrates a framework in which a structural mesh system 100 may be used to provide structural support to a tissue construct 102 that may be anastomosed to a host vasculature 110. The structural mesh system 100 may include a mesh casing 104 and suturable cuff 108, which are discussed in more detail below. The structural mesh system 100 may encase and provide structural support to the tissue construct 102.

The term “tissue construct” may refer to any tissue construct or organ that comprises cells and extracellular matrix material, preferably with interpenetrating vasculature. In some examples, the tissue construct 102 may be a multi-layered tissue construct that includes from two to eight cell layers. As illustrated in FIG. 1B, the tissue construct may be assembled from cell aggregates or spheroids. The tissue construct may comprise adipose tissue. The tissue construct 102 may include a vascular channel 118 that facilitates the passage of a fluid, for example blood or another bodily fluid, through the tissue construct 102. The tissue construct 102 may have any size and geometry that may be prepared by 3D printing or another fabrication method.

The structural mesh system 100 may provide structural or mechanical support to the tissue construct 102, for example, after it has been anastomosed to a host vasculature and perfused with blood from the host. Referring again to FIG. 1A, the structural mesh system 100 may include a mesh casing 104, a capsule 106, and a suturable cuff 108. The mesh casing 104 may be shaped to partially or fully encase the tissue construct 102. For example, the mesh casing may 104 be designed such that the tissue construct 102 may be inserted or placed into the mesh casing 104.

The mesh casing 104 may be used bare, or it may be embedded in a capsule 106. The capsule 106 can be made with any type of biological hydrogel, synthetic hydrogel, and/or any soft material compatible with the homeostasis and overall function of the tissue construct. In one example, the capsule 106 may include gelatin, for example, gelatin crosslinked with transglutaminase. Other materials for the capsule 106 may include gelatin-methacrylate, hyaluronic acid, fibrin, and/or collagen. The capsule 106 may also be porous and/or allow for the passage of oxygen, nutrients, and/or molecules from outside of the capsule 106 into the tissue construct 102. The capsule 106 may conform to the shape of the mesh casing 104, for example, to stay in place on the mesh casing 104.

The suturable cuff 108 may be any device or structure capable of connecting the tissue construct 102 to the host vasculature 110, for example, in an in vivo or an in vitro application. Additionally or alternatively, the suturable cuff 108 may be any device or structure capable of securing the mesh casing 104 to the tissue construct 102. However, the mesh casing 104 may be connected to the tissue construct 102 without the use of a suturable cuff 108. For example, the tissue construct 102 may be encased by the mesh casing 104 without a suturable cuff 108 as described below.

Referring back to FIG. 1B, in one example, the suturable cuff 108 may comprise a hollow body having an anastomotic end 112, an anchoring end 114, and one or more lumens 116, where each lumen extends through the hollow body from a proximal opening at the anastomotic end 112 to a distal opening at the anchoring end 114. In some examples, the suturable cuff 108 may include two (or more) lumens 116, and thus two (or more) proximal openings at the anastomotic end 112 and two (or more) distal openings at the anchoring end 114. The suturable cuff 108 may be as described, for example, in PCT/US23/62916 to Reynolds et al., entitled “Suturable Cuff, Method of Integrating a Tissue Construct with a Host Organ or Vasculature, and Method of Making a Suturable Cuff,” which is hereby incorporated by reference.

Alternatively, the tissue construct 102 may be encased by the mesh casing 104 in an in vitro application. The tissue construct 102 and mesh casing 104 may be disposed, for example, in a culture medium. The tissue construct 102 may not be anchored to a suturable cuff, and the mesh casing 104 may allow for perfusion of the tissue construct 102 from the culture environment.

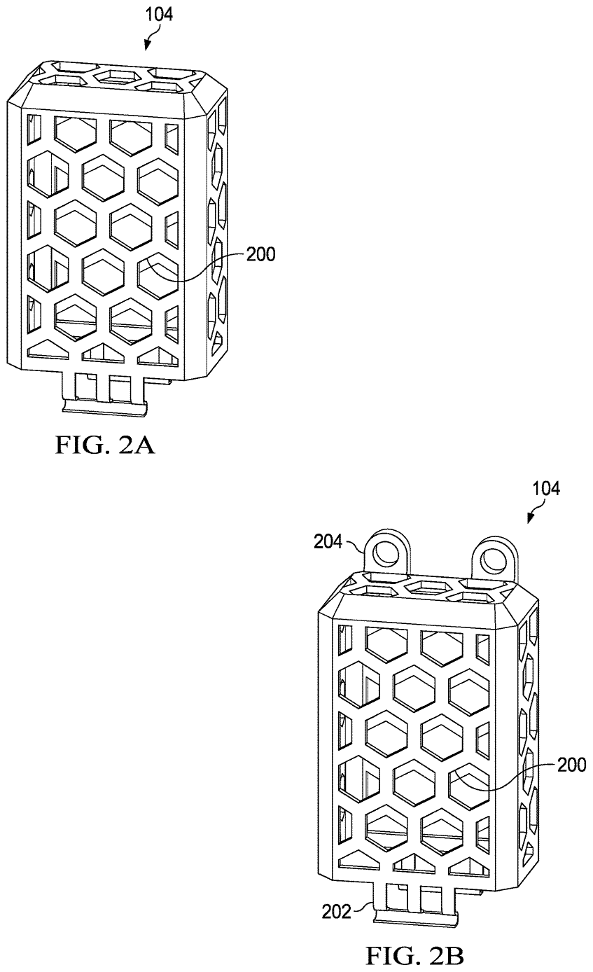

FIGS. 2A and 2B illustrate exemplary designs of the mesh casing 104. The mesh casing 104 may include pores 200 that may allow, for example, the transport of oxygen, nutrients, and/or any molecules necessary for the survival and development of the tissue construct 102 through the mesh casing 104. Additionally or alternatively, the pores 200 in the mesh casing 104 may allow communication between the tissue construct 102 with the surrounding host tissue. The pores 200 in the mesh casing 104 may be arranged in, for example, a pattern. The pores 200 may be, for example, honeycomb or hexagonal, circular, square, ovular, triangular, diamond, pentagonal, or octagonal in shape. The pores 200 may, for example, be regular, symmetrical, irregular, or asymmetrical in shape. The mesh casing 104 may have pores 200 with different shapes. The pores 200 may have dimensions ranging from submicrometers to millimeters. For example, the pores may have any dimension suitable to allow for nutrients, oxygen, and/or other molecules to pass through the pores 200 in the mesh casing 104.

The mesh casing 104 may comprise a mesh material having a stiffness sufficient for the mesh casing 104 to provide structural support while the tissue construct sustains internal pressure from blood circulation. The mesh material may be a biocompatible and/or biodegradable material. In one example, the mesh material may be a polymer, such as a natural or synthetic polymer. Suitable mesh materials may include silicone, metals such as stainless steel or titanium, or polymers such as polyacrylate, polycaprolactone or poly-lactic-co-glycolic acid. To provide the requisite structural support, the mesh casing 104 may be rigid or semi-rigid. For example, the mesh material may have a stiffness that is greater than that of fat tissue, for example stiffer than 2 kPa. The material may have a Young's modulus in the range of 1500-2500 MPa, for example, of 2080 MPa. The rigidity of the mesh casing may depend on the Young's modulus of the mesh material and the geometry of the mesh casing (including the pores 200 of the mesh casing). The mesh material may be rigid or stiff enough to provide structural or mechanical support to the tissue construct 102, when the tissue construct 102 sustains internal pressure from blood circulation when anastomosed to the host vasculature 110. For example, the mesh material may be rigid or stiff enough to support the tissue construct 102 when the tissue construct 102 sustains an internal blood pressure of up to 150 mmHg. For example, the mesh material may support the tissue construct 102 while the tissue construct 102 experiences a blood pressure of 100 mmHg (arterial) and between 5 mmHG to 10 mmHg (venous).

The mesh casing 104 may be fabricated via 3D printing, molding, or micromachining, and/or may be assembled from existing and/or commercially available parts. For example, the mesh casing 104 may be printed on a 3D printer, for example, the Form3 by Formlabs. The mesh material may be biocompatible for in vivo integration. In one example, the mesh casing 104 may be printed out of Form3 biomed resin, or another biocompatible resin. Additionally or alternatively, the mesh material may be biodegradable. Additionally or alternatively, the mesh material may be opaque or optically transparent. Additionally or alternatively, the mesh casing 104 may include a functional coating. For example, the mesh casing 104 may be coated with a material that includes one or more agents with anti-inflammatory and/or anti-fibrotic properties. Additionally or alternatively, the coating may have drug eluting properties. For example, the mesh casing 104 may be coated with medication, for example, a slow release medication. Additionally or alternatively, the mesh casing 104 may be coated with antibiotic, anti-inflammatory, anti-fibrotic, pro-angiogenic, and/or chemotherapy medication.

As shown in FIGS. 2A and 2B, the mesh casing 104 may be shaped like a rectangular prism. The mesh casing 104 may be open at one end to allow for insertion of the tissue construct 102. Additionally or alternatively, the mesh casing 104 may include a clip 202 to secure the mesh casing to the suturable cuff 108 and form the structural mesh system 100. Additionally or alternatively, the mesh casing 104 may include suture anchors or loops 204, as shown in FIG. 2B, for securing the mesh casing on to, for example, tissue expanders. The clip 202 and/or the suture loops 204 may formed as a unitary, singular structure with the mesh casing 104.

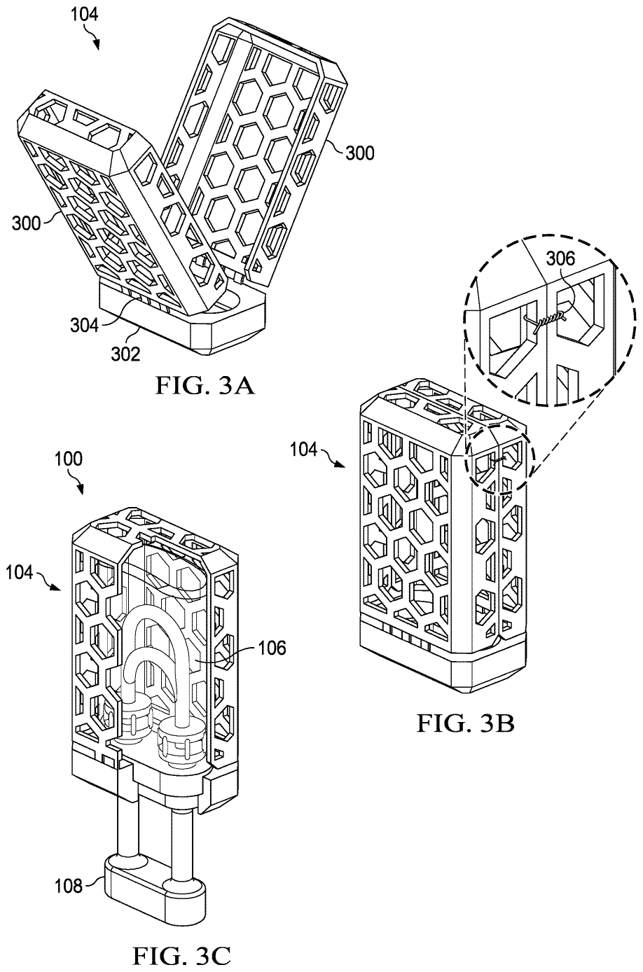

FIGS. 3A-3D illustrate another example of the mesh casing 104 where the mesh casing 104 may comprise a base 302 and two halves 300. Each of the two halves 300 may be connected to the base 302 by a respective hinge 304 on either side of the base. The hinges 304 may be flexible. Each half 300 and its respective hinge 304 may be disposed on opposite sides of the base 302 such that, when closed, as shown in FIG. 3B, the halves 300 meet and form an enclosed casing to contain the tissue construct. This functionality may be referred to as a clamshell mechanism. The mesh casing 104 may surround the suturable cuff 108. In one example, the suturable cuff 108 may be inserted into an opening in the base 302, as shown in FIG. 3C. Additionally or alternatively, the suturable cuff 108 may extend into the mesh casing 104. The mesh casing halves 300 may surround or encase the tissue construct 102 when closed together. For example, the two halves 300 may close around the tissue construct 102. The two halves 300 may be held closed, surrounding the tissue construct 102, with, for example, a suture 306 as shown in FIG. 3B, or another mechanism, such as prongs or clips.

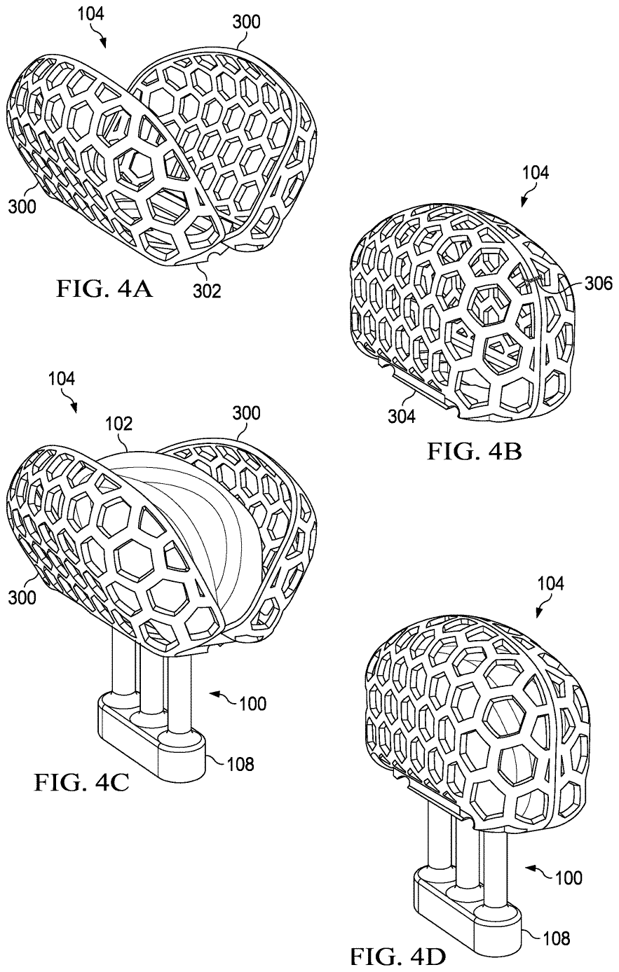

FIGS. 4A-4D illustrate a mesh casing 104 accommodating an organ-specific geometry. In this example, the mesh casing 104 is shaped as and fabricated to protect a kidney-shaped tissue construct 102. Alternatively, the mesh casing 104 may be shaped like different bodily organ. The mesh casing 104 may have any of various geometries. In particular, it may be designed such that it can accommodate the form factor of an organ-inspired biological construct.

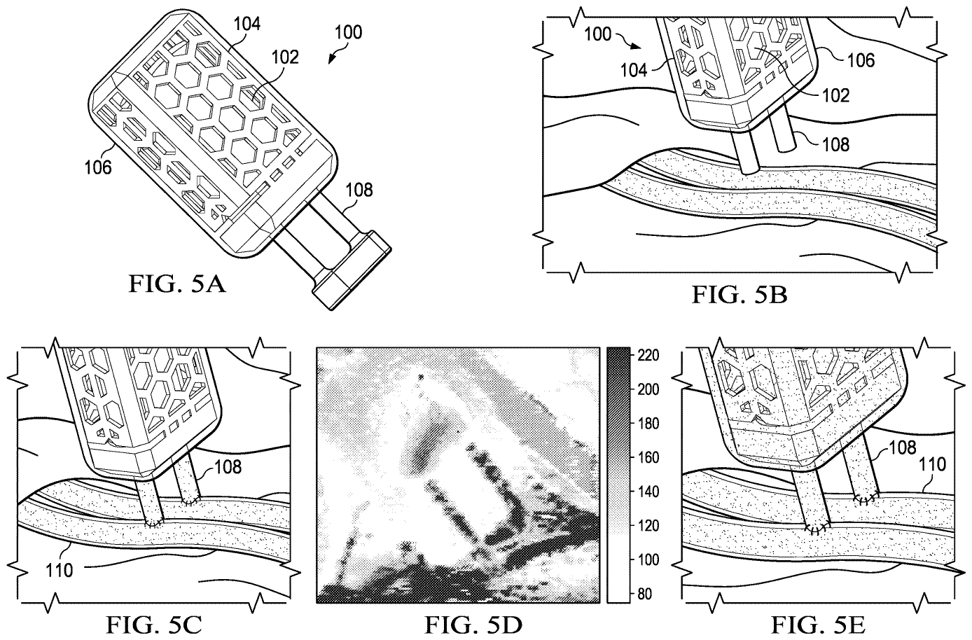

FIGS. 5A-5E illustrate application of the structural mesh system 100 on a tissue construct 102. An adipose tissue construct 102 may be assembled, for example, using adipocyte-containing spheroids (or organ building blocks or OBBs shown in FIG. 1B) and packed into a dense matrix. The cellular matrix may serve as a support for the templating of vascular conduits using, for example, the sacrificial writing into functional tissue (SWIFT) technique. The tissue construct 102 may then be connected to the suturable cuff 108 (e.g., see FIG. 1B). After, for example, two days of in vitro perfusion, the tissue construct 102 may be extracted, inserted into the mesh casing 104 of the structural mesh system 100, and encapsulated in a gelatin capsule 106 (FIG. 5A). Upon removal of the basal support of the cuffs 108 (FIG. 5B), tubular projections from the suturable cuff 108 (the pair of silicone legs) may be anastomosed to the, for example, a femoral artery and vein of a host organism 110 (FIG. 5C and FIG. 1A). FIG. 5D shows blood flow through the tissue being measured with doppler imaging, and the integrity of the tissue construct 102 following, for example, two days of implantation can be assessed prior to tissue harvesting (FIG. 5E). The structural mesh system 100 may prevent the inflation and eventually the destruction of the tissue construct 102 under blood flow.

In one example, the method of connecting the tissue construct 102 to a host organ 110 or vasculature 110 may include providing the suturable cuff 108. The tissue construct 102 that includes a vascular channel is anchored to the anchoring end 114 of the suturable cuff 108. During the anchoring, the vascular channel may be positioned to be in fluid communication with the distal opening of the hollow body. If the suturable cuff 108 includes two or more lumens 116 and accordingly two or more distal openings at the anchoring end 114, the vascular channel may be positioned to be in fluid communication with the two or more distal openings, thereby facilitating, during in vivo use, fluid (e.g., blood) flow into and out of the tissue construct. It is noted that objects described in this disclosure as being “in fluid communication” with each other (e.g., the vascular channel and the distal opening) are positioned with respect to each other such that a fluid can flow between and/or through the objects, in one or both directions.

The suturable cuff 108 with the anchored tissue construct 102 is then inserted into the mesh casing 104, and possibly also the capsule 106, of the structural mesh system 100. In some examples, the capsule 106 is applied to the mesh casing 104 immediately prior to implantation of the structural mesh system 100 into a body of a patient. Integration of the suturable cuff 108 and the mesh casing 104 with the tissue construct 102 is discussed further below.

The structural mesh system 100 with the tissue construct 102 may then be inserted into a body of a patient and positioned at a desired in vivo location for connection to the host organ or vasculature 110. One or more body vessels 110 (e.g., vein(s), artery(ies), duct(s), hollow organ(s) etc.) may be sewn, sutured, or otherwise anastomosed to the anastomotic end 112 of the suturable cuff 108, such that the body vessel(s) are in fluid communication with the proximal opening(s) of the hollow body. For example, the body vessel(s) may be anastomosed to the anastomotic end 112 via an end-to-end connection or a side-to-end ‘T’ connection. The one or more body vessels may form part or all of the host organ or vasculature. After anastomosis, the suturable cuff 108 and the anchored tissue construct 102 may be perfused with blood or another bodily fluid from the patient delivered through the body vessel(s).

The above procedure may be used in various surgical applications such as reconstructive surgery and organ transplants (e.g., kidney transplants). The anchoring or securing of the tissue construct 102 to the anchoring end 114 of the suturable cuff 108 may take place during or after fabrication of the tissue construct 102. As described above, the anchoring end 114 of the suturable cuff 108 includes one or more anchoring features, and thus the anchoring may be understood as partially or fully embedding the one or more anchoring features within the tissue construct 102. In one example, the one or more anchoring features may be inserted into the tissue construct 102 post-fabrication (of the tissue construct). Due to the configuration of the anchoring features, the insertion may result in securing of the tissue construct 102 to the suturable cuff. In addition, during the insertion, the suturable cuff 108 may be positioned such that the distal opening of the hollow body is in fluid communication with the vascular channel.

Alternatively, the anchoring may take place during fabrication of the tissue construct 102. In such an example, the tissue construct 102 may be formed around the anchoring feature(s), such that the one or more anchoring features are partially or fully embedded in the tissue construct 102. Preferably, during fabrication, a vascular channel formed within the tissue construct 102 is positioned to be in fluid communication with the one or more distal openings of the hollow body. For example, printing or another fabrication method that allows for both embedding of the anchoring feature(s) into the tissue construct 102 and alignment of the vascular channel(s) with the distal opening(s) may be employed, such that the tissue construct 102 is both anchored to the suturable cuff and is perfusable after implantation into a patient's body.

The mesh casing 104 may be placed around the tissue construct either before, during, or after construction of the tissue construct 102. For example, the fully fabricated tissue construct 102 may be inserted into the mesh casing 104 after the tissue construct 102 is fully formed and before placement into a host body. Alternatively, the mesh casing 104 may be connected to the suturable cuff 108 before or during fabrication of the tissue construct 102 such that the tissue construct 102 is at least partially formed or fabricated while encased in the mesh casing 104. Additionally or alternatively, the placement of the capsule 106 around the mesh casing 104 may, for example, happen after fabrication of the tissue construct 102 and after placement of the tissue construct 102 inside of the mesh casing 104. For example, the capsule 106 may be placed around the mesh casing 104 already encasing the fully fabricated tissue construct 102 just before the tissue construct and structural mesh system 100 are placed inside a host body.

Alternatively, the mesh casing 104 may be used without the suturable cuff 108. In such embodiment, the tissue perfusable channels may be directly anastomosed to the host vasculature or fluid duct without a biological or synthetic cuff interface, provided that the tissue construct can sustain handling and suturing to the host animal.

The subject matter of the disclosure may also relate to the following aspects:

A first aspect relates to a structural mesh for a tissue construct, the structural mesh comprising: a mesh casing shaped to partially or fully enclose a tissue construct, the mesh casing comprising: a mesh material having a stiffness sufficient for the mesh casing to provide structural support while the tissue construct sustains internal pressure from blood circulation; and pores configured to allow for transport of oxygen and nutrients between the tissue construct and a surrounding environment.

A second aspect relates to the structural mesh of the preceding aspect, wherein the mesh casing is configured for in vivo use.

A third aspect relates to the structural mesh of any preceding aspect, wherein the mesh casing is configured for in vitro use

A fourth aspect relates to the structural mesh of any preceding aspect, wherein the mesh material is biodegradable.

A fifth aspect relates to the structural mesh of any preceding aspect, wherein the mesh material comprises a polymer.

A sixth aspect relates to the structural mesh of any preceding aspect, wherein the mesh material is selected from the group consisting of: include acrylate resins, silicone, metals such as stainless steel or titanium, or polymer such as polycaprolactone or poly-lactic-co-glycolic acid.

A seventh aspect relates to the structural mesh of any preceding aspect, wherein the mesh material has a Young's modulus in the range of 1500-2500.

An eighth aspect relates to the structural mesh of any preceding aspect, wherein the mesh casing comprises a base and two halves, wherein each of the two halves is connected to the base by a respective hinge, the halves pivotable about their respective hinges to enclose the tissue construct.

A ninth aspect relates to the structural mesh of any preceding aspect, wherein the mesh casing is shaped like a rectangular prism.

A tenth aspect relates to the structural mesh of any preceding aspect, wherein the mesh casing is shaped like a bodily organ.

An eleventh aspect relates to the structural mesh of any preceding aspect, wherein the mesh casing is shaped like a kidney.

A twelfth aspect relates to the structural mesh of any preceding aspect, wherein the pores are hexagonal in shape.

A thirteenth aspect relates to the structural mesh of any preceding aspect, wherein the pores in the mesh are circular, square, ovular, triangular, diamond, pentagonal, or octagonal in shape.

A fourteenth aspect relates to the structural mesh of any preceding aspect, wherein the mesh casing comprises a loop or anchor for suturing to tissue expanders.

A fifteenth aspect relates to a structural mesh system, the system comprising: a tissue construct including a vascular channel; and a mesh casing partially or fully enclosing the tissue construct.

A sixteenth aspect relates to the structural mesh system of the preceding aspect further comprising a suturable cuff anchored to the tissue construct and configured for integration with one or more body vessels.

A seventeenth aspect relates to the structural mesh system of any preceding aspect, wherein the mesh casing is configured for in vivo use.

An eighteenth aspect relates to the structural mesh system of any preceding aspect, wherein the mesh casing is configured for in vitro use.

A nineteenth aspect relates to the structural mesh system of any preceding aspect, wherein the suturable cuff comprises a hollow body having an anastomotic end, an anchoring end, and one or more lumens, each lumen extending through the hollow body from a proximal opening at the anastomotic end to a distal opening at the anchoring end.

A twentieth aspect relates to the structural mesh system of any preceding aspect, wherein the vascular channel of the tissue construct is in fluid communication with the distal opening.

A twenty-first aspect relates to the structural mesh system of any preceding aspect, wherein the mesh casing comprises a mesh material having a Young's modulus in the range of 1500-2500 MPa.

A twenty-second aspect relates to the structural mesh system of any preceding aspect, wherein the mesh material is a polymer.

A twenty-third aspect relates to the structural mesh system of any preceding aspect, wherein the mesh material is biodegradable.

A twenty-fourth aspect relates to the structural mesh system of any preceding aspect, wherein the mesh material is rigid or semi-rigid to provide structural support to the tissue construct sustaining internal pressure from blood circulation.

A twenty-fifth aspect relates to the structural mesh system of any preceding aspect, wherein the mesh casing comprises a base and two halves, wherein each of the two halves is connected to the base by a respective hinge, the halves being pivotable about their respective hinges to enclose the tissue construct.

A twenty-sixth aspect relates to the structural mesh system of any preceding aspect, wherein the mesh casing is shaped like a rectangular prism.

A twenty-seventh aspect relates to the structural mesh system of any preceding aspect, wherein the mesh casing is shaped like a bodily organ.

A twenty-eighth aspect relates to the structural mesh system of any preceding aspect wherein the mesh casing is shaped like a kidney.

A twenty-ninth aspect relates to the structural mesh system of any preceding aspect wherein the tissue construct comprises vascularized adipose tissue.

A thirtieth aspect relates to the structural mesh system of any preceding aspect, wherein the mesh casing comprises pores for transportation of oxygen and nutrients between the tissue construct and a surrounding environment.

A thirty-first aspect relates to the structural mesh system of any preceding aspect, wherein the pores are hexagonal in shape.

A thirty-second aspect relates to the structural mesh system of any preceding aspect, wherein the pores are circular, square, ovular, triangular, diamond, pentagonal, or octagonal in shape.

A thirty-third aspect relates to the structural mesh system of any preceding aspect, wherein the mesh casing comprises a loop or anchor for suturing to a tissue expander.

A thirty-fourth aspect relates to the structural mesh system of any preceding aspect, wherein the system further comprises a gelatin capsule surrounding the mesh casing.

A thirty-fifth aspect relates to the structural mesh system of any preceding aspect, wherein the gelatin capsule is crosslinked with transglutaminase.

A thirty-sixth aspect relates to the structural mesh system of any preceding aspect, wherein the gelatin capsule comprises a hydrogel.

A thirty-seventh aspect relates to a structural mesh system comprising: a tissue construct including a vascular channel; a mesh casing partially or fully enclosing the tissue construct; and a suturable cuff anchored to the tissue construct and configured for integration with one or more body vessels, wherein the structural mesh system is disposed in a body of a patient, and wherein one or more body vessels are in fluid communication with the suturable cuff.

A thirty-eighth aspect relates to the structural mesh system of the preceding aspect, wherein the suturable cuff comprises a hollow body having an anastomotic end, an anchoring end, and one or more lumens, each lumen extending through the hollow body from a proximal opening at the anastomotic end to a distal opening at the anchoring end.

A thirty-ninth aspect relates to a method of supporting a tissue construct in an in vivo or an in vitro application, the method comprising: providing a tissue construct including a vascular channel; placing the tissue construct inside of a mesh casing, the mesh casing surrounding the tissue construct; inserting the mesh casing and the tissue construct into a body of a patient or into a culture medium; perfusing the tissue construct with blood or another bodily fluid, wherein, during the perfusion, the mesh casing provides support to the tissue construct to withstand a pressure of the blood or other bodily fluid being perfused into the tissue construct.

A fortieth aspect relates to the method of the preceding aspect, further comprising, prior to inserting the mesh casing and the tissue construct into the body of the patient, anchoring the tissue construct to a suturable cuff configured for integration with one or more body vessels.

A forty-first aspect relates to a method of supporting a tissue construct in a host organ or vasculature, the method comprising: providing a suturable cuff comprising a hollow body having an anastomotic end, an anchoring end, and one or more lumens, each lumen extending through the hollow body from a proximal opening at the anastomotic end to a distal opening at the anchoring end; anchoring a tissue construct including a vascular channel to the anchoring end of the suturable cuff, the vascular channel being positioned to be in fluid communication with the distal openings; placing the tissue construct inside of a mesh casing, the mesh casing surrounding the tissue construct; anchoring the tissue construct to the suturable cuff; inserting the mesh casing and suturable cuff with the anchored tissue construct into a body of a patient; anastomosing one or more body vessels to the anastomotic end of the suturable cuff, whereby the one or more body vessels are in fluid communication with the proximal openings; perfusing the suturable cuff and the anchored tissue construct with blood or another bodily fluid from the patient; and wherein, during the perfusion, the mesh casing provides support to the tissue construct to withstand a pressure of the blood or other bodily fluid being pumped through the tissue construct.

A forty-second aspect relates to the method of the preceding aspect further comprising, prior to inserting the mesh casing and suturable cuff into the body of the patient, placing a gelatin capsule around the mesh casing to provide additional support to the tissue construct.

A forty-third aspect relates to the method of any preceding aspect, wherein the mesh casing includes a coating with anti-inflammatory or anti-fibrotic properties.

A forty-fourth aspect relates to the method of any preceding aspect, wherein the mesh casing includes a coating with drug eluting properties.

Each component may include additional, different, or fewer components. The system 100 may be implemented with additional, different, or fewer components. To clarify the use of and to hereby provide notice to the public, the phrases “at least one of <A>, <B>, . . . and <N>” or “at least one of <A>, <B>, . . . <N>, or combinations thereof” or “<A>, <B>, . . . and/or <N>” are defined by the Applicant in the broadest sense, superseding any other implied definitions hereinbefore or hereinafter unless expressly asserted by the Applicant to the contrary, to mean one or more elements selected from the group comprising A, B, . . . and N. In other words, the phrases mean any combination of one or more of the elements A, B, . . . or N including any one element alone or the one element in combination with one or more of the other elements which may also include, in combination, additional elements not listed. Unless otherwise indicated or the context suggests otherwise, as used herein, “a” or “an” means “at least one” or “one or more.”

While various embodiments have been described, it will be apparent to those of ordinary skill in the art that many more embodiments and implementations are possible. Accordingly, the embodiments described herein are examples, not the only possible embodiments and implementations.

In addition to the features mentioned in each of the independent aspects enumerated above, some examples may show, alone or in combination, the optional features mentioned in the dependent aspects and/or as disclosed in the description above and shown in the figures.

1. A structural mesh for a tissue construct, the structural mesh comprising:
a mesh casing shaped to partially or fully enclose a tissue construct, the mesh casing comprising:
a mesh material having a stiffness sufficient for the mesh casing to provide structural support while the tissue construct sustains internal pressure from blood circulation; and
pores configured to allow for transport of oxygen and nutrients between the tissue construct and a surrounding environment.

2-3. (canceled)

4. The structural mesh of claim 1, wherein the mesh material is biodegradable.

5. (canceled)

6. The structural mesh of claim 1, wherein the mesh material is selected from the group consisting of: polyacrylate, polycaprolactone, poly-lactic-co-glycolic acid, silicone, and a metal.

7. The structural mesh of claim 1, wherein the mesh material has a Young's modulus in the range of 1500-2500 MPa.

8. The structural mesh of claim 1, wherein the mesh casing comprises a base and two halves, wherein each of the two halves is connected to the base by a respective hinge, the halves being pivotable about their respective hinges to enclose the tissue construct.

9-11. (canceled)

12. The structural mesh of claim 1, wherein the pores are hexagonal in shape.

13. The structural mesh of claim 1, wherein the pores in the mesh are circular, square, ovular, triangular, diamond, pentagonal, or octagonal in shape.

14. (canceled)

15. A structural mesh system, the system comprising:
a tissue construct including a vascular channel; and
a mesh casing partially or fully enclosing the tissue construct.

16. The structural mesh system of claim 15 further comprising a suturable cuff anchored to the tissue construct and configured for integration with one or more body vessels.

17-18. (canceled)

19. The structural mesh system of claim 16, wherein the suturable cuff comprises a hollow body having an anastomotic end, an anchoring end, and one or more lumens, each lumen extending through the hollow body from a proximal opening at the anastomotic end to a distal opening at the anchoring end.

20. The structural mesh system of claim 19, wherein the vascular channel of the tissue construct is in fluid communication with the distal opening.

21. The structural mesh system of claim 15, wherein the mesh casing comprises a mesh material having a Young's modulus in the range of 1500-2500 MPa.

22. (canceled)

23. The structural mesh system of claim 15, wherein the mesh material is biodegradable.

24. (canceled)

25. The structural mesh system of claim 15, wherein the mesh casing comprises a base and two halves, wherein each of the two halves are connected to the base by a respective hinge, the halves being pivotable about their respective hinges to enclose the tissue construct.

26-29. (canceled)

30. The structural mesh system of claim 15, wherein the mesh casing comprises pores for transportation of oxygen and nutrients between the tissue construct and a surrounding environment.

31-32. (canceled)

33. The structural mesh system of claim 15, wherein the mesh casing comprises a loop or an anchor for suturing to a tissue expander.

34. The structural mesh system of claim 15, wherein the system further comprises a gelatin capsule surrounding the mesh casing.

35-36. (canceled)

37. The structural mesh system of claim 15, the system further comprising:
a suturable cuff anchored to the tissue construct,
wherein the structural mesh system is disposed in a body of a patient, and
wherein one or more body vessels are in fluid communication with the suturable cuff.

38. (canceled)

39. A method of supporting a tissue construct in an in vivo or an in vitro application, the method comprising:
providing a tissue construct including a vascular channel;
placing the tissue construct inside of a mesh casing, the mesh casing surrounding the tissue construct;
inserting the mesh casing and the tissue construct into a body of a patient or into a culture medium;
perfusing the tissue construct with blood or another bodily fluid,
wherein, during the perfusion, the mesh casing provides support to the tissue construct to withstand a pressure of the blood or other bodily fluid being perfused into the tissue construct.

40. The method of claim 39 further comprising, prior to inserting the mesh casing and the tissue construct into the body of the patient, anchoring the tissue construct to a suturable cuff configured for integration with one or more body vessels.

41-44. (canceled)
